(19) United States
(12) Patent Application Publication
(10) Pub. No.: US 2020/0188868 A1
(43) Pub. Date: Jun. 18, 2020

(54) METHOD AND APPARATUS FOR LINING A CHAMBER

(71) Applicant: 4D Pharma León S.L.U., Leon (ES)

(72) Inventors: Christophe CARITE, Yorkshire (GB); Jean-Pascal ZAMBAUX, Bouliac (FR)

(21) Appl. No.: 16/729,038

(22) Filed: Dec. 27, 2019

Related U.S. Application Data
(63) Continuation of application No. PCT/IB2018/054749, filed on Jun. 27, 2018.

(30) Foreign Application Priority Data
Jun. 28, 2017 (GB) 1710355.7

(51) Int. Cl. B01F 15/00; C12M 1/00; C12M 1/04; C12M 1/34; C12M 1/12

(52) U.S. Cl. CPC B01F 15/00837; C12M 23/26; C12M 37/00; C12M 41/40; C12M 23/24

(57) ABSTRACT

The present invention provides a method and kit which can be used to prevent or reduce contaminants in a clean production environment. The method involves providing a processing apparatus having a chamber, and providing a liner within the chamber, wherein a negative pressure is applied to the interior of the chamber to retain the liner against the interior wall of the chamber.

### CROSS-REFERENCE

This application is a continuation of International Application No. PCT/IB2018/054749, filed Jun. 27, 2018, which claims the benefit of Great Britain Application No. 1710355.7, filed Jun. 28, 2017, all of which are hereby incorporated by reference in their entirety.

### FIELD OF THE INVENTION

The present invention relates to methods and apparatus for reducing contaminants within clean processing environments.

### BACKGROUND TO THE INVENTION

There are many processes which must be performed in clean environments which are free of, or at least contain acceptably low levels of contaminants. For example, in the development and production of pharmaceutical or biotechnological products, the exclusion of contaminants is critical, in order to ensure patient safety and compliance of those products with regulatory requirements.

Such products typically contain component parts, for example, the active ingredient or agent itself, excipients with which the active ingredient may be formulated, and packaging for the finished pharmaceutical product. During the preparation of finished products, the active ingredient and excipients will typically be subjected to a number of processing steps in order to obtain a finished product having the required properties.

Contaminants in the preparation of pharmaceutical and biotechnological products may originate from a range of sources. For example, the active ingredient or agent and excipients may each originate from different commercial sources which individually could introduce contaminants into the production line. Additionally, although the conduct and dress code of operatives on the production line for pharmaceutical and biotechnological products is strictly controlled, the inadvertent introduction of contaminants by operatives cannot be excluded.

Additionally, there is a risk of cross-contamination, for example where processing apparatus is used to produce different products. More specifically, processing apparatus (especially that suitable for industrial scale use) is often costly and thus, for entities engaged in the production of a range of pharmaceutical or biotechnological products, it is necessary for the same apparatus to be employed in the production of different products.

When the use of such processing apparatus to produce a first product has been completed and that apparatus is being prepared for use to produce a second product, it is necessary (and indeed, in most countries, a regulatory requirement) for that apparatus to be thoroughly cleaned to remove all traces of the first product prior to being used to manufacture the second product.

The need to ensure the removal of all traces of component parts used, and products produced, in prior production runs of the apparatus is especially acute for certain types of product, for example high potency products (where even trace amounts of an active agent from a prior production run could have an observable effect on patients who are administered products produced in subsequent production runs) or bacterial products (where single organisms could contaminate and reproduce in products produced in subsequent production runs).

A further complicating factor is that, in many cases, processing apparatus employed in the production of pharmaceutical and biotechnological products is complex, with numerous component parts, and thus the consistent, thorough cleaning of such apparatus may be challenging.

A range of techniques has been developed to sterilise pharmaceutical and biotechnological processing apparatus, including the use of gamma irradiation, heat-treatment by autoclave, steam cleaning or exposure of the apparatus to hydrogen peroxide. While such techniques are successful in inactivating potentially harmful bacteria, they typically do not result in the physical removal of particulate contaminants. Further, processing down-time is required in order for the sterilisation techniques to be performed.

There remains a need for techniques which can enable the convenient removal of all kinds of contaminants from a zone in which the processing of pharmaceutical or biotechnological products is carried out.

### SUMMARY OF THE INVENTION

Thus, according to a first aspect of the present invention, there is provided a method of lining a chamber comprising providing a chamber having an interior defined by a chamber wall, the chamber wall being provided with a plurality of air permeable openings, providing a flexible liner within the chamber, the flexible liner having an outer surface and an inner surface, applying a negative pressure into the interior of the chamber via the plurality of the openings in the chamber wall and maintaining the negative pressure within the chamber for a period of time to retain the liner against the chamber wall, reducing or stopping the negative pressure applied to the interior of the chamber to enable removal of the liner from the chamber.

The application of a liner to the interior wall of the chamber via negative pressure effectively prevents contaminants within the chamber from coming into contact with the inside of the liner, and also effectively prevents contaminants within the liner from coming into contact with the interior of the chamber wall. Upon completion of an operation or process being conducted in the chamber, the negative pressure applied to the interior of the chamber can be reduced or removed, thus enabling convenient and rapid removal of the liner and any contaminants and products within the liner. In embodiments of the invention, the operation performed in the chamber may be a mixing or blending operation.

A further advantage of the method of the first aspect of the invention is that the application of a negative pressure to the interior of the chamber has the effect of drawing the liner towards the interior wall of the chamber and maintaining it against that interior wall. This prevents folds or peaks being formed in the liner which could otherwise be problematic, for example where the chamber is being used for a mixing operation, as unmixed components could collect within folds in the liner resulting in non-uniform mixing of the components, or peaks of the liner could project into the interior of the chamber and become snarled in mixing apparatus employed within the chamber.

The process of the invention has wide applicability in terms of the types of chamber with which it can be used. For example, the chamber could be the chamber in a mixer (e.g. a Y-cone mixer, a cube-mixer, a double cone mixer, an agitator mixer, a mixer employing a magnetic stirrer, a helical mixer, a turbine mixer, a propeller mixer, an in-line mixer, a planetary mixer or a sigma blade mixer), milling apparatus (e.g. a cutter mill, an end runner mill, an edge runner mill, a roller mill, a hammer mill, a vibration mill, a pin mill, a ball mill or a fluid energy mill), a separator, a sieve, a granulator (e.g. a fluidized bed granulator, a wet granulator, a shear granulator, an oscillating granulator, a high speed mixer/granulator, a Collette-Gral granulator, a Freund granulator, a slugger or a roller compactor), a dryer (e.g. a tunnel drier, a rotary drier, a fluidised bed drier, a vacuum oven, a vacuum tumbling dryer, a microwave, a radiant heat dryer, a drum dryer, a spray dryer or a rotary atomiser), a direct compressor, a rotary tablet press, a tablet coating pan, a tablet coating compressor, an encapsulator, a spheroniser, a pelletiser, a baffled or unbaffled tank, a hopper in which active pharmaceutical ingredients and/or excipients are stored, a steriliser, a washer, a lyophiliser, a lyophilisation tray, a packaging line, or any other chamber used in a pharmaceutical or biotechnological processing apparatus.

With the method of the present invention, components which are disposed within the chamber, during use of the chamber in a processing apparatus, may be disposable. For example, in embodiments in which the chamber is a chamber in a mixer, some or all of the component parts of the mixing apparatus may be disposable.

Additionally or alternatively, the chamber could be a room or other enclosed space (such as a fume cupboard, isolator, or clean room) in which processing apparatus such as that mentioned in the preceding paragraphs is employed, or in which other pharmaceutical or biotechnological products are handled or stored or processes (e.g. analytical, investigational or other research activities) conducted.

As will be appreciated, the method of the present invention is not only applicable in the pharmaceutical and biotechnological processing fields; the subject matter of the invention may find application more widely, for example in the food processing, electronics production (e.g. semiconductor production), satellite production, automotive component production or aerospace component production fields.

Regardless of the specific application to which the method of the present invention is employed, it will be recognised that the method enables processing apparatus to be re-used in different production runs with the risk of cross-contamination being minimised. Additionally or alternatively, the method of the present invention may include the step of filling the chamber with one or more component parts of the product being produced, and/or with one more component parts of the processing apparatus. For the avoidance of doubt, the term ‘filling’ as used herein does not necessarily mean that the interior of the chamber is completely filled, but that a quantity of material is provided into the chamber.

Additionally, or alternatively, in the method of the present invention, the liner may be a single layer or have a multi-layer construction, for example, comprising 2, 3, 4, 5, 6, 7, 8 or more layers. In multi-layer constructions, the material from which each layer is prepared may be the same as one another, or may be different from the material from which at least one of the other layers is prepared.

One, some, or all of the layers of the liner may be formed from a plastic material, for example the plastic material may comprise plasticised or unplasticised polyvinyl chloride (PVC), latex, polyethylene (e.g. LDPE, MDPE, HDPE), polypropylene, polyetheretherketone (PEEK), polyester (e.g. BoPET), polytetrafluoroethylene (e.g. Teflon), nylon, perfluoroalkoxy (PFA), silicone rubber, bioplastics such as polycaprolactone, cellulose or polylactic acid, or blends thereof.

Depending on the intended application of the method, the liner may be intended for single-use, such that once the process conducted in the chamber is completed, the bag is removed from the chamber and disposed of. Alternatively, the liner may be removed from the chamber, emptied if needed, optionally cleaned (and optionally sterilised), and returned to the chamber for re-use.

The liner may be of any thickness provided that it meets the requirements of serving as an effective barrier to contaminants and retains an acceptable degree of flexibility for the desired application. In particular, the liner may have a thickness of about 0.1 μm to about 50 μm, about 0.2 μm to about 20 μm, about 0.5 μm to about 10 μm or about 1 μm to about 5 μm. Alternatively, for example where a more robust liner is required, the thickness of the liner may range from about 10 μm to about 500 μm, about 100 μm to about 400 μm or about 150 μm to about 350 μm.

The air permeability of the liner should be chosen such that the liner is securely retained against the chamber wall. Thus, the liner may have an oxygen transmission rate (OTR, in units of cm³·m⁻²·d⁻¹) of less than about 5000, less than about 2000, less than about 1000, less than about 500, less than about 200, less than about 100, less than about 50, less than about 20 or less than about 10.

Additionally or alternatively, in processing methods where the interior of the chamber contains moisture, it will typically be undesirable for that moisture to pass through the liner upon the application of a negative pressure to the interior of the chamber. Thus, the liner may have a water vapour transmission rate (WVTR, in units of g·m⁻²·d⁻³) of less than about 200, less than about 100, less than about 50, less than about 20, less than about 10 or less than about 5.

A further advantage of the present invention is that it is applicable to the processing of both solid and liquid component parts. For example, the lined chamber may be used to mix powder, for example as part of a double-cone mixer. Alternatively, the lined chamber may used to mix liquid component parts, for example as part of a stirred tank. Once the mixing step has been concluded, and the mixed composition has been removed, the negative pressure applied to the interior of the chamber can be reduced or removed enabling the liner to be removed from the chamber, and leaving the chamber contaminant free.

During the steps of applying and maintaining the negative pressure within the interior of the chamber, the strength of the negative pressure applied may be varied. For example, the negative pressure applied to the interior of the chamber relative to the interior of the liner may be less than 0, less than about −2, less than about −5, less than about −10, less than about −20, less than about −50, less than about −100, less than about −200, less than about −500, or less than about −1000 Pa.

The negative pressure applied to the interior of the chamber may then be altered, for example it may be increased or decreased. For example, the negative pressure may be reduced, by about 10% or more, by about 20% or more, by about 30% or more, by about 50% or more, or by about 70% or more. Alternatively, the negative pressure may be increased by about 10% or more, by about 20% or more, by about 30% or more, by about 50% or more, or by about 70% or more for the remainder of the step of applying and maintaining negative pressure into the interior of the chamber.

Additionally or alternatively, the step of reducing or stopping the negative pressure applied to the interior of the chamber to enable removal of the liner from the chamber may involve a reduction in the strength of the negative pressure applied to the interior of the chamber by about 50% or more, about 60% or more, about 70% or more, about 80% or more, about 90% or more, or about 95% or more.

The construction of the liner can take any suitable form. Preferably, the liner may be in the form of a bag defining an interior and an exterior. In such an embodiment, the exterior of the bag defines the outer surface of the liner, while the interior of the bag defines the inner surface of the liner.

In embodiments where the liner is in the form of a bag, the bag may comprise an access opening. The access opening provides access to the interior of the bag, for example for filling/emptying of the bag, to enable component parts of the processing apparatus to be located in the bag, or to pump air or another gas into the bag. In embodiments in which the chamber is large (e.g. a fume cupboard, isolator or a clean room), the access opening may provide access into the chamber for one or more operatives.

The access opening may be provided by making an incision in the wall of the bag to provide a flap, or it may be provided by making a shaped aperture in the wall of the bag. The incision or aperture may be formed by cutting the wall of the bag.

Closure means may be provided to close the access opening. For example, a lid may be provided. The closure means may be formed of a flexible material (for example the same material as the walls of the bag). Alternatively, the closure means may be formed of a rigid material. The closure means may be connected to the bag, e.g. via a hinge, cord or other connector type. Alternatively, the closure means may be separable from the bag.

An example of a closure means for use in the method of the present invention comprises a lid having a flange or skirt around its perimeter, wherein the flange or skirt can be adhesively sealed to a region of the outer surface of the bag surrounding the access opening.

In alternative embodiments, the access opening may be provided with means to engage the closure means. For example, the access opening may be provided with a collar around its perimeter which can receive the closure means. In such embodiments, the collar may be provided with a flange which is joined to the interior or exterior of the bag (e.g. via welding or gluing) in the region adjacent to the access opening. The closure means may be coupled to the collar using means known to the skilled person, for example via a threaded screw-fit, friction fit, snap-fit or the like.

Alternatively or additionally, the bag may be provided with a frangible seal which closes the access opening so that, during transport and fitting of the bag, the interior of the bag remains closed. When the operator wishes to use the bag, the frangible seal can be broken so that the interior of the bag can be accessed.

The access opening may be provided with coupling means to enable it to be connected to a component part of the processing apparatus. For example, if the bag is used to line a mixing chamber, and the mixing blades are to be inserted into the chamber via the access opening, the access opening can be provided with coupling means to enable it to be connected to the mixing apparatus, thus effectively closing the bag for the duration of the mixing step.

Additionally or alternatively, the access opening may be provided with coupling means to enable it to be connected to a dust extractor, a steriliser or other processing means. Examples of such coupling means are disclosed in International Patent Application No. WO2010/007288.

A further example of apparatus that may be connected to the coupling means is a pump. The pump may be used to deliver positive pressure into the bag, for example to inflate the bag prior to the intended process commencing. Additionally or alternatively, the pump may deliver negative pressure into the bag, for example, to remove air and/or particulate matter from the bag upon completion of the intended process.

The coupling means may take any form provided that they enable the bag to be connected to a component part of the processing apparatus. For example, the access opening may be provided with a collar around its perimeter which can be coupled to the component part of the processing apparatus. In such a case, the collar may be provided with a flange which is joined to the interior or exterior of the bag (e.g. via welding or gluing) in the region adjacent to the access opening. The component part of the processing apparatus may be coupled to the collar using means known to the skilled person, for example via a threaded screw-fit, friction fit, snap-fit or the like.

In embodiments of the invention in which the access opening is provided with a collar around its perimeter, the collar may be used to engage closure means and/or coupling means.

As described above, the bag may comprise a single access opening. Alternatively, the bag may comprise a plurality of access openings as described above, for example 2, 3, 4, 5, 6, 7, or 8 access openings. In embodiments where a plurality of access openings are provided, one may be provided with closure means and another may be provided with coupling means. Alternatively, the same access opening may be provided with both closure means and coupling means.

In addition to access openings, the bag may be provided with one or more ports which enable information to be obtained regarding properties of the contents of the bag. Such ports may be designed to receive probes which can be used to monitor, for example, temperature, pH, oxygen concentration, optical density and/or pressure. For example, the bag may comprise 1, 2, 3, 4, 5, 6, 7, or 8 ports.

The method of the present invention may further comprise the step of sterilising the interior of the chamber and/or the environment defined by the inner wall of the liner. This sterilisation step may be conducted prior to, simultaneously with and/or following the step of reducing or stopping the negative pressure applied into the interior of the chamber. Depending on the intended application of the bag, the interior of the bag may be pre-sterilised such that the interior is sterile prior to use.

The liner used in the method of the present invention may comprise gloves welded, adhered or formed therein. These gloves enable an operator to handle and work on materials within the chamber without having to gain access to the interior of the chamber, thus minimising the risk of contaminants entering the chamber.

One advantage of the method of the present invention is that it may be used with liners or chambers of any size. For example, the liner and/or chamber may have a capacity of at least about 1 litre, at least about 2 litres, at least about 3 litres, at least about 4 litres, at least about 5 litres, at least about 10 litres, at least about 15 litres, at least about 20 litres, at least about 50 litres, at least about 100 litres, at least about 200 litres, or at least about 500 litres.

In embodiments in which the chamber is large (e.g. where it is a fume cupboard isolator, or clean room), the liner and/or chamber may have a capacity of about 5 m³ or greater, about 10 m³ or greater, about 20 m³ or greater or about 30 m³ or greater optionally up to about 70 m³ or lower, or about 100 m³ or lower.

The liner may be shaped and sized so that its external profile corresponds to the shape and size of the interior of the chamber.

The strength of the negative pressure applied to the interior of the chamber relative to the interior of the linee, for example by a vacuum pump, may vary depending on the intended application and how tightly the liner is required to be retained against the chamber wall. It has been found that a relatively low negative pressure is sufficient to effectively retain the liner against the chamber wall and thus the negative pressure applied to the interior of the chamber may be from less than about 0, less than about −2, less than about −5, less than about −10, less than about −20, less than about −50, less than about −100, less than about −200, less than about −500 or less than about −1000 Pa.

The amount of negative pressure required may be balanced against the size of the air permeable openings in the chamber wall. The openings may be of any size and shape provided that they permit sufficient negative pressure through the wall of the chamber to retain the liner against the chamber wall when the negative pressure is applied, whilst at the same time providing enough support for the liner to maintain its integrity and position within the chamber.

For example, the air permeable openings may each have an area of about 0.01 cm², about 0.02 cm², about 0.05 cm², about 0.1 cm², about 0.2 cm², or about 0.5 cm² to about 1 cm², about 1.5 cm² or about 2 cm². The air permeable openings may each have the same area, or at least one of the air permeable openings may have a different area to at least one of the other air permeable openings Additionally or alternatively, the air permeable openings may have a circular, oval, square, rectangular, polygonal or other shape.

The negative pressure may be applied to the interior of the chamber by any means known to those skilled in the art, for example the negative pressure may be applied by sucking air out of the chamber through the plurality of openings using a vacuum pump. The chamber may be provided with vacuum channels via which the negative pressure may be applied to the interior of the chamber.

Additionally or alternatively, a positive pressure may be applied to the interior of the liner and/or the environment defined by the inner surface of the liner to balance the negative pressure applied through the wall of the chamber. The positive pressure may be applied by a pump which is used to inflate the liner. The positive pressure may be used to create the negative pressure in the interior of the chamber relative to the interior of the liner, thus forcing air out of the chamber through the plurality of openings.

One advantage of the present invention is the broad applicability of the inventive method, which can be used with a broad range of chambers, as discussed above. Any chamber can be employed, provided that it has a wall comprising a plurality of openings to allow the application of a negative pressure to the interior of the chamber. It is envisaged that existing processing apparatus can be easily adapted to enable use of the method of the present invention, simply by replacing the chamber(s) present in existing processing apparatus with chambers that have interior chamber walls comprising a plurality of air permeable openings.

Thus, according to a further aspect of the present invention, there is provided a chamber for use in the method described herein.

According to a still further aspect of the present invention, there is provided a liner (e.g. a bag) for use in the method described herein.

A further aspect of the present invention is a kit which enables operation of the method described herein. Specifically, the kit comprises a) a chamber having an interior defined by a chamber wall, the chamber wall being provided with a plurality of air permeable openings, and b) a flexible liner comprising an access opening, the flexible liner having an external profile which corresponds to the shape and size of the interior of the chamber. The chamber and the flexible liner may comprise the features described above, with reference to the first aspect of the present invention.

### BRIEF DESCRIPTION OF THE DRAWINGS

By way of example, one or more embodiments of the present invention will now be described with reference to the accompanying drawings in which:

FIG. 1a is a cross-sectional view of a chamber and liner for use in accordance with a first embodiment of the present invention;

FIG. 1b is a cross-sectional view of the apparatus of FIG. 1a when a negative pressure has been applied to the interior of the chamber;

FIG. 1c is a cross-sectional view of the apparatus of FIGS. 1a and 1b when component parts of a product being mixed in the chamber have been placed inside the liner;

FIG. 1d is a cross-sectional view of the apparatus of FIGS. 1a to 1c when a mixing apparatus has been inserted into the interior of the liner;

FIG. 1e is a cross-sectional view of the apparatus of FIGS. 1a to 1d when the negative pressure has been removed from the interior of the chamber;

FIG. 1f is a cross-sectional view of the apparatus of FIGS. 1a to 1e when the liner is being removed from the interior of the chamber; and

FIG. 2 is a view of a liner for use in accordance with a second embodiment of the present invention.

### DETAILED DESCRIPTION OF THE DRAWINGS

### Example 1—Cone Mixer

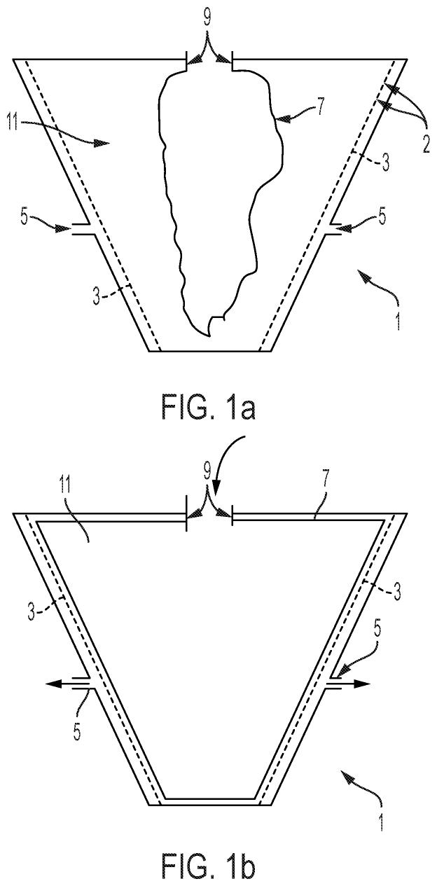

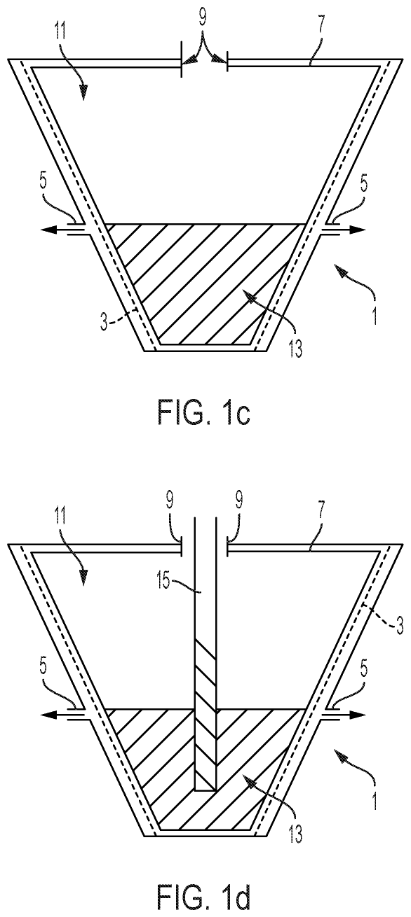

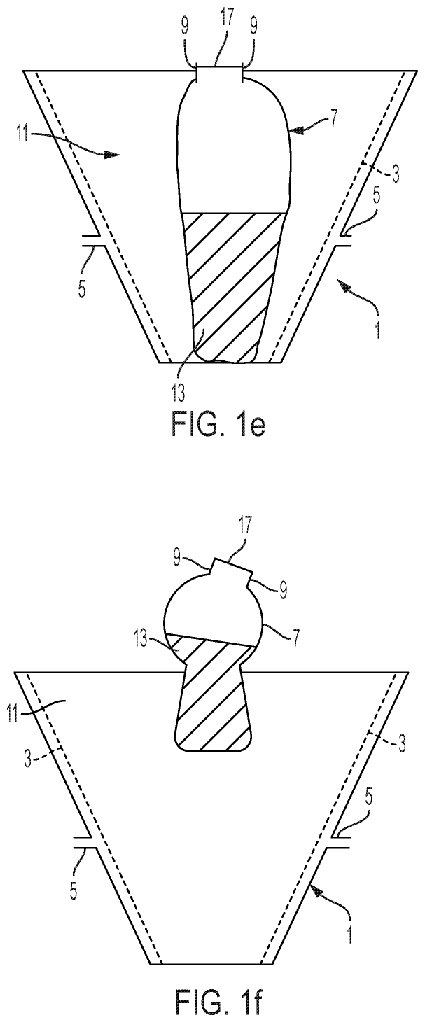

FIGS. 1a to 1f show an arrangement of component parts of a cone mixer which can be used in a method according to a first aspect of the present invention. The component parts include a liner and chamber for use in the method according to the first aspect of the present invention, and also comprise a kit according to a further aspect of the present invention.

As shown in FIG. 1a, the method comprises providing a chamber 11 which has an interior defined by a chamber wall 3. The chamber wall 3 is provided with a plurality of air permeable openings 2. The chamber 11 is part of a cone mixer 1. Outer walls of the cone mixer 1 include vacuum channels 5 which can be connected to a vacuum pump (not shown) that can be used to apply a negative pressure to the interior of the chamber 11 through the air permeable openings 2. The method further comprises providing a flexible liner 7, comprising a bag, the flexible liner 7 having an outer surface and an inner surface. The flexible liner 7 is located within the chamber 11, and includes an access opening 9 comprising a collar.

As shown in FIG. 1b, air is pumped into the liner 7 via the access opening defined by collar 9 using a pump (not shown). Simultaneously, negative pressure is applied into the interior of the chamber 11 via vacuum channels 5 and the plurality of air permeable openings 2 in the interior chamber wall 3. The liner 7 is shaped such that its exterior surface corresponds to the shape of the interior wall 3 of the cone mixer 1. The negative pressure is applied and maintained within the chamber 11 for a period of time which causes the liner 7 to be drawn to and retained against the interior wall 3 of the cone mixer 1.

As shown in FIG. 1c, the flow of air into the liner 7 is stopped. Material 13 which is to be mixed in the chamber is then placed inside the liner 7 via the access opening 9. Negative pressure continues to be applied to the interior of the chamber 11 to retain the liner 7 against the interior wall 3 of the chamber of the cone mixer 1.

As shown in FIG. 1d, mixing apparatus 15 (which may be disposable or reusable) is then inserted into the interior of the liner 7 via access opening 9. The mixing apparatus 15 is then operated until mixing of the material 13 is complete.

As shown in FIG. 1e, the mixing apparatus 15 is then removed from the liner 7, and the liner 7 is closed with rigid closure 17. The application of negative pressure into the interior of the chamber 11 is then stopped such that the liner 7 is no longer retained against the inner wall 3 of the chamber 11 of the cone mixer 1. This allows the liner 7 to be removed from the chamber 11 of the cone mixer 1 as shown in FIG. 1f.

Following extraction of the liner 7 from the chamber 11, the cone mixer 1 is immediately ready for use in another mixing operation without the need to clean or sterilise the interior of the mixing chamber 11.

A replacement liner 7 can then be inserted into the interior of the chamber, and the process outlined above can be repeated. Alternatively, in the event that the same material is to be processed, the liner 7 used in the process described above can be re-used.

### Example 2—Fume Cupboard

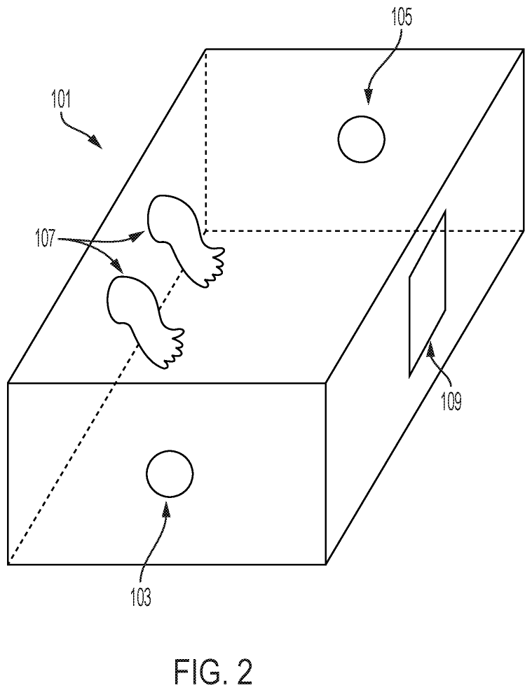

An alternative arrangement is shown in FIG. 2. In that arrangement a flexible liner 101 is constructed to have an outer profile which corresponds to the shape of a fume cupboard (not shown). The walls of the fume cupboard are provided with openings (not shown) through which air can pass. The liner is positioned within the fume cupboard, and a negative pressure is applied to the interior of the fume cupboard through those openings to retain the liner 101 against the walls of the cupboard.

The liner 101 is additionally provided with inlet 103, outlet 105 and gloves 107. A window formed of transparent material 109 is also provided to enable observers to view the interior of the liner 101. The regions of the fume cupboard walls (not shown) adjacent to the window 109, inlet 103 and outlet 105 are cut away and not provided with openings.

The negative pressure that has been applied to the interior of the fume cupboard is then reduced or stopped to allow the liner 101 to be removed from the fume cupboard.

It will be appreciated that the embodiments shown in the figures are by way of example only, and that alterations or modifications may be made within the scope of the invention as defined in the following claims.

1.-21. (canceled)

22. A method comprising:
(a) providing a chamber with a flexible liner displaced therein, wherein the chamber comprises a wall with an air permeable opening, wherein the flexible liner comprises an outer surface and an inner surface, wherein the flexible liner has a thickness of from 0.1 μm to 500 μm, and wherein the flexible liner has an air permeable opening;
(b) applying a negative pressure through the air permeable opening of the chamber and maintaining the negative pressure within the chamber for a period of time, thereby retaining the flexible liner against the wall of the chamber; and
(c) applying a positive pressure to an interior of the flexible liner via the air permeable opening in the flexible liner to inflate the flexible liner while applying the negative pressure into the interior of the chamber.

23. The method of claim 22, wherein the flexible liner has an oxygen transmission rate of less than 5000 cm³·m⁻²·d⁻¹.

24. The method of claim 22, wherein the flexible liner has a water vapor transmission rate of less than 200 g·m⁻²·d⁻³.

25. The method of claim 22, wherein the flexible liner is of a material selected from the group consisting of: polyvinylchloride, latex, polyethylene, polypropylene, polyetheretherketone, polyester, nylon, polytetrafluoroethylene, silicone rubber, cellulose, polylactic acid, and perfluoroalkoxy alkane.

26. The method of claim 22, wherein the flexible liner further comprises an access opening.

27. The method of claim 26, further comprising disposing a bacterial composition for pharmaceutical use into the interior of the flexible liner through the access opening.

28. The method of claim 27, further comprising performing a process on the bacterial composition in the interior of the flexible liner.

29. The method of claim 28, wherein the process comprises mixing the bacterial composition disposed in the interior of the chamber.

30. The method of claim 29, further comprising inserting a mixing apparatus into the access opening of the flexible liner.

31. The method of claim 26, wherein the flexible liner comprises a lid to close the access opening.

32. The method of claim 31, wherein the lid comprises an adhesive.

33. The method of claim 26, wherein the access opening comprises a frangible seal.

34. The method of claim 26, wherein the access opening comprises a collar around a perimeter of the access opening.

35. The method of claim 22, wherein the chamber is a mixer or a blender.

36. A method of lining a chamber comprising:
(a) providing a clean room, an isolator, or a fume cupboard having an interior defined by a wall of the clean room, the isolator, or the fume cupboard, wherein the wall has an air permeable opening;
(b) introducing a flexible liner into the clean room, the isolator, or the fume cupboard, wherein the flexible liner comprises an outer surface and an inner surface;
(c) applying a negative pressure into the interior of the clean room, the isolator, or the fume cupboard via the air permeable opening and maintaining the negative pressure for a period of time to retain the flexible liner against the wall of the clean room, the isolator, or the fume cupboard; and
(d) reducing or stopping the negative pressure applied to the interior of the clean room, the isolator, or the fume cupboard to enable removal of the flexible liner.

37. The method of claim 36, wherein the flexible liner is of a material selected from the group consisting of: polyvinylchloride, latex, polyethylene, polypropylene, polyetheretherketone, polyester, nylon, polytetrafluoroethylene, silicone rubber, cellulose, polylactic acid, and perfluoroalkoxy alkane.

38. The method of claim 36, wherein the flexible liner is inserted into a fume cupboard.

39. The method of claim 38, wherein the flexible liner further comprises gloves attached to a wall of the flexible liner.

40. The method of claim 36, wherein the flexible liner has an oxygen transmission rate of less than 5000 cm³·m⁻²·d⁻¹.

41. The method of claim 36, wherein the flexible liner has a water vapor transmission rate of less than 200 g·m⁻²·d⁻³.

42. The method of claim 36, wherein the flexible liner comprises a window formed of a transparent material.

43. A method of reducing cross contamination of a bacterial composition in a chamber, comprising culturing a bacterial composition for pharmaceutical use disposed in an interior of a flexible liner, wherein the flexible liner is disposed within the chamber on a wall of the chamber by negative pressure applied through an air permeable opening on the wall of the chamber, wherein the air permeable opening of the chamber has an area of from 0.01 cm² to 2 cm², and wherein the bacterial composition does not contact the wall of the chamber.

44. The method of claim 43, wherein the flexible liner is not permeable to material in the chamber outside of the flexible liner.

45. The method of claim 44, wherein the material in the chamber outside of the flexible liner does not contact the bacterial composition disposed in the interior of the flexible liner.

46. An apparatus comprising:
(a) a chamber having a capacity of at least 1 liter, wherein the chamber comprises a wall with an air permeable opening, wherein the air permeable opening has an area of from 0.01 cm² to 2 cm²;
(b) a vacuum pump operatively coupled to the air permeable opening configured to generate a pressure of less than 100 Pa;
(c) a flexible liner disposed on the wall of the chamber, wherein the flexible liner comprises a water vapor transmission rate of less than 200 g·m⁻²·d⁻³ and a thickness of from 0.1 μm to 500 μm; and
(d) a bacterial composition for pharmaceutical use disposed in the interior of the flexible liner.
